(19) United States
(12) Patent Application Publication
(10) Pub. No.: US 2011/0218406 A1
(43) Pub. Date: Sep. 8, 2011

(54) Visual Display For Medical Monitor

(75) Inventor: Tashriq Hussain, Parker, CO (US)

(73) Assignee: Nellcor Puritan Bennett LLC, Boulder, CO (US)

(21) Appl. No.: 12/717,238

(22) Filed: Mar. 4, 2010

(51) Int. Cl. A61B 5/00; G09G 5/00

(52) U.S. Cl. 600/300; 345/660

(57) ABSTRACT

The present disclosure describes a visual display for a medical monitor that may present a plurality of visualizations representative of physiological measurements. The visualizations may differ depending on whether the corresponding physiological parameter is within an expected range. The visualizations may include text, waveforms, images, video, and 3-dimensional representations. The visualizations may include different sizes and display properties such as colors, font types, and font styles.

### BACKGROUND

The present disclosure relates generally to medical monitors and, more particularly, to visual displays for medical monitors.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of healthcare, caregivers (e.g., doctors and other healthcare professionals) often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

Medical monitoring devices, for example, pulse oximetry monitors, typically include a screen display that presents a variety of information such as a pulse rate, arterial oxygen saturation, a wavefom, and so forth. Traditionally, the presented information tracks a current patient state and is usually updated after a certain time interval, for example, every few seconds. In addition to monitoring a patient's physiological characteristics, a pulse oximeter or other patient monitor may alert a caregiver when certain physiological conditions are recognized. For example, a normal range for a particular physiological parameter of a patient may be defined by setting low and/or high threshold values for the physiological parameter, and an alarm may be generated when a detected value of the physiological parameter is outside the normal range. Medical monitor screen displays typically have a small footprint, that is, the display may be sized to fit into a small desktop monitor or similar device. Accordingly, certain alarms and visuals of interest may not be quickly and easily noticed or read.

### BRIEF DESCRIPTION OF THE DRAWINGS

Advantages of the disclosed techniques may become apparent upon reading the following detailed description and upon reference to the drawings in which:

FIG. 1 illustrates a perspective view of a medical monitoring system, in accordance with an embodiment of the present technique;

FIG. 2 illustrates a medical monitor display screen, in accordance with an embodiment of the present technique;

FIG. 3 illustrates a second medical monitor display screen, in accordance with an embodiment of the present technique;

FIG. 4a illustrates a third medical monitor display screen, in accordance with an embodiment of the present technique;

FIG. 4b illustrates a fourth medical monitor display screen, in accordance with an embodiment of the present technique;

FIG. 5 illustrates a fifth medical monitor display screen, in accordance with an embodiment of the present technique; and

FIG. 6 illustrates a flowchart in accordance with an embodiment of the present technique.

### DETAILED DESCRIPTION

One or more embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain medical monitoring devices, a display may be used to present patient-related information such as pulse, arterial oxygen saturation (SpO₂), waveforms (e.g., plethysmographs , electrocardiograms, electroencephalograms), and so forth. Some measurements, for example, a low pulse value, may require the attention of a clinician. It would be advantageous for the clinician to readably visualize information, such as the pulse rate, from various locations within the patient's room, including the patient's bedside. Accordingly, the disclosed embodiments include visualization systems and techniques that aid in attracting the attention of a clinician and that provide for increased readability and accessibility of medical information.

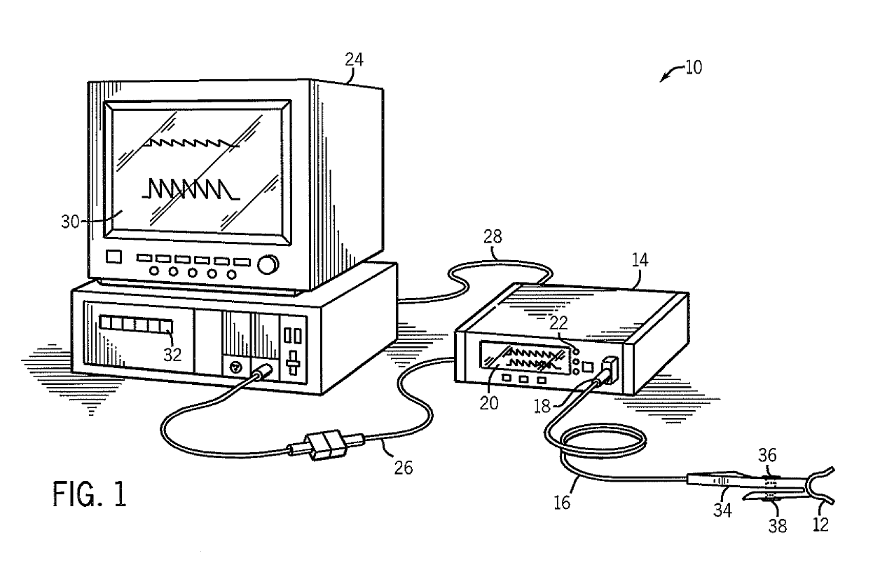

With this in mind, and turning now to the figures, FIG. 1 depicts an embodiment of a patient monitoring system 10 that may display a variety of patient information readable from considerable distances and viewing angles, thereby providing the clinician with an increase in freedom of movement and situational awareness. Although the embodiment of the patient monitoring system 10 illustrated in FIG. 1 relates to photoplethysmography, the system 10 may be configured to obtain a variety of medical measurements with a suitable medical sensor. For example, the system 10 may, additionally or alternatively, be configured to determine patient temperature, transvascular fluid exchange volumes, tissue hydration, blood flow, cardiovascular effort, glucose levels, level of consciousness, total hematocrit, hydration, electrocardiography, electroencephalograpy, or any other suitable physiological parameter. The system 10 includes a sensor 12 that is communicatively coupled to a patient monitor 14 via a cable 16 through and a sensor port 18. Additionally, the monitor 14 includes a monitor display 20 configured to display information regarding the physiological parameters, information about the system, and/or alarm indications. The monitor 14 may include various input components 22, such as knobs, switches, keys and keypads, buttons, etc., to provide for operation and configuration of the monitor. The monitor 14 also includes a processor that may be used to execute code such as code for implementing the techniques discussed herein.

Furthermore, to upgrade conventional operation provided by the monitor 14 to provide additional functions, the monitor 14 may be coupled to a multi-parameter patient monitor 24 via a cable 26 connected to a sensor input port or via a cable 28 connected to a digital communication port. In addition to the monitor 14, or alternatively, the multi-parameter patient monitor 24 may be configured to calculate physiological parameters and to provide a central display 30 for the visualization of information from the monitor 14 and from other medical monitoring devices or systems. The multi-parameter monitor 24 includes a processor that may be configured to execute code. The a multi-parameter monitor 24 may also include various input components 32, such as knobs, switches, keys and keypads, buttons, etc., to provide for operation and configuration of the a multi-parameter monitor 24. In addition, the monitor 14 and/or the multi-parameter monitor 24 may be connected to a network to enable the sharing of information with servers or other workstations.

The sensor 12 may be any sensor suitable for detection of any physiological parameter. The sensor 12 may include optical components (e.g., one or more emitters and detectors), acoustic transducers or microphones, electrodes for measuring electrical activity or potentials (such as for electrocardiography), pressure sensors, motion sensors, temperature sensors, etc. In one embodiment, the sensor 12 may be configured for photo-electric detection of blood and tissue constituents. For example, the sensor 12 may be a pulse oximetry sensor, such as those available from Nellcor-Puritan Bennett LLC. As shown in FIG. 1, the sensor 12 may be a clip-type sensor suitable for placement on an appendage of a patient, e.g., a digit, an ear, etc. In other embodiments, the sensor 12 may be a bandage-type sensor having a generally flexible sensor body to enable conformable application of the sensor to a sensor site on a patient. In yet other embodiments, the sensor 12 may be secured to a patient via adhesive (e.g., in an embodiment having an electrode sensor) on the underside of the sensor body or by an external device, such as headband or other elastic tension device. In yet other embodiments, the sensor 12 may be configurable sensors capable of being configured or modified for placement at different sites (e.g., multiple tissue sites, such as a digit, a forehead of a patient, etc.).

In one embodiment, the sensor 12 may include a sensor body 34 having an emitter 36 for emitting light at certain wavelengths into a tissue of a patient and a detector 38 for detecting the light after it is reflected and/or absorbed by the blood and/or tissue of the patient. In such an embodiment where the sensor 12 is a pulse oximetry sensor or other photo-electric sensor, the emitter 36 may be configured to emit one or more wavelengths of light, e.g., red and infrared (IR), such as through light emitting diodes (LEDs) or other light sources. The detector 38 may include photo-detectors for detecting the wavelengths of light reflected or transmitted through blood or tissue constituents of a patient and converting the intensity of the received light into an electrical signal.

In certain embodiments, the sensor 12 may be a wireless sensor 12. Accordingly, the wireless sensor 12 may establish a wireless communication with the patient monitor 12 and/or the multi-parameter patient monitor 24 using any suitable wireless standard. By way of example, the wireless module 26 may be capable of communicating using one or more of the ZigBee standard, WirelessHART standard, Bluetooth standard, IEEE 802.11x standards, or MiWi standard.

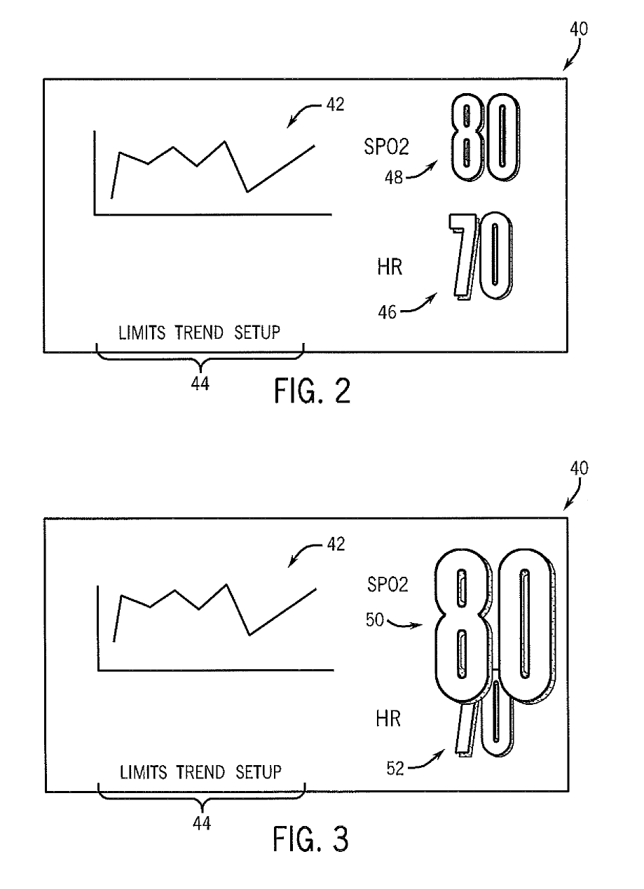

Turning to FIG. 2, the figure depicts an embodiment of a display screen 40 that may be displayed by the monitor display 20 and/or the central display 30 from FIG. 1. The display screen 40 may be used to present any number of medically-related visualizations, including waveforms, text, and other multimedia (e.g., video, 3D graphics, images, animations). In the depicted embodiment, visualizations including a waveform 42, text 44, heart rate measurement 46, and SpO₂ measurement 48 are presented. The medical monitors 14 and/or 24 are configured to continuously refresh the display screen 40 with the latest visualizations, including sensor 12 measurements, after a certain periodic time interval has elapsed. For example, the display screen 40 may periodically update the presented visualizations after a time interval of approximately 5 milliseconds, 500 milliseconds, 1 second, 5, seconds, or 10 seconds. Accordingly, the display screen 40 is a dynamic display screen capable of continuously updating the visualizations 42, 44, 46, 48 and/or presenting new visualizations. Indeed, input devices included in the medical system 10, such as the input components 22, 32 shown in FIG. 1, may allow a clinician to configure the display screen 40 so as to present any number of visualizations.

In certain embodiments, a plurality of audible and visual alarms may be configured based on certain patient events and measurement thresholds. For example, a SpO₂ alarm may be configured to activate when the patient's measured SpO₂ is outside of certain threshold values (e.g., upper and lower threshold values). A clinician may configure the threshold values depending on any number of factors such as patient age (e.g., neonate, child, adult), clinical condition (e.g., infarction, cardiac arrest, respiratory illness), clinical history, and so forth. Any number of alarms may be configured, for example, alarms that activate based on heart rate, temperature, respiration rate, blood pressure, expiratory CO2, and so forth. Certain alarms may include system alarms configured to activate based on system events and measurements. Accordingly, system events and measurements such as sensor cable 16 disconnections, low sensor 12 battery power, loss of wireless signal, and so forth, may be used to activate the system alarms. The activation of one or more alarms may in turn initiate certain audible tones and visualizations as described in greater detail with respect to FIGS. 3-5 below.

FIG. 3 depicts an embodiment of certain visualizations presented by the display screen 40 when an alarm, such as the SpO₂ alarm, has been activated. As mentioned above, the SpO₂ alarm may have been activated due to the SpO₂ patient measurement crossing a threshold value, for example, a current SpO₂ value lower than approximately 95. Low SpO₂ values may require the attention of a clinician. Accordingly, the alarm may aid in procuring the attention of a clinician by, for example, initiating an audible tone, and/or displaying an enhanced SpO₂ measurement 50. The SpO₂ measurement 50 has a larger font size, may have a different font type, a different font style, and/or a different color than the SpO₂ measurement 48 depicted in FIG. 2. For example, the font height may increase from an initial height of approximately 10%, 20%, 30%, 40% of the screen 40 height to a vertical height of approximately 60%, 70%, 80%, 90% of the screen 40 height. In certain embodiments, the font width may increase proportionally in order to maintain the same font aspect ratio. In other embodiments, the font aspect ratio may change, for example by increasing the font width disproportionably to the font height, so as to improve readability. In certain embodiments, the font type (e.g., Helvetica, Arial, Teletype) may change to different font type, the font style may change to a different font style (e.g., boldface, underline, shadowed), and the font color may change to a different font color (e.g., red, orange, yellow). The background color of all or part of the screen 40 may also change so as to improve readability and/or attract attention. All display properties (e.g., font height, font width, font type, font style, font color) of the SpO₂ measurement 50 and screen 40 are configurable by the user. Indeed, the display properties of all visualizations described herein, such as the visualizations described in the FIGS. 2-5, are configurable by the user.

Additionally or alternatively, a measurement within normal range (i.e., at or within of the alarm upper and lower thresholds), may be displayed less prominently in order to increase visibility of the alarm measurement 50. Accordingly, the measurement 52 may be may be displayed less prominently, for example, by reducing the font size, increasing the font opacity (i.e., making the displayed value more transparent), changing the color, changing the font type, and/or changing the font style. For example, the font height may decrease from an initial height of approximately 10%, 20%, 30%, 40% of the screen 40 height to a vertical height of approximately 5%, 10%, 15%, 20% of the screen 40 height. In certain embodiments, the font width may decrease proportionally in order to maintain the same font aspect ratio. In other embodiments, the font aspect ratio may change in order to increase the noticeability of the alarm and improve readability. In certain embodiments, the font type, font style, and font color may also change. Other enhanced or enlarged displays may include, for example, a video display, an image display, or a 3-dimensional (3D) display. Such displays may be enlarged to cover a larger area of the screen 40 and/or enhanced by changing colors, changing aspect ratios, displaying textual annotations, and so forth.

The display of enhanced or enlarged measurements such as the SpO₂ measurement 50 and heart rate measurement 52 increases the readability and noticeability of the alarm condition. Indeed, by presenting visualizations such as those depicted in FIG. 3, the alarm value (e.g., SpO₂ measurement 50) can be noticed and read across a considerable distance (e.g., 10 ft., 15 ft., 20 ft. away) and through a wide range of viewing angles (e.g., 25°, 35°, 45°). Indeed, the example visualizations such as the SpO₂ measurement 50 and heart rate measurement 52 of FIG. 3 may allow the clinician to easily notice and read the patient's alarm measurement from almost all locations in the patient's room or outside a doorway to the patient's room. Such an improvement in noticeability and readability allows for the clinician to concentrate on patient treatment instead of on screen display 40 adjustments.

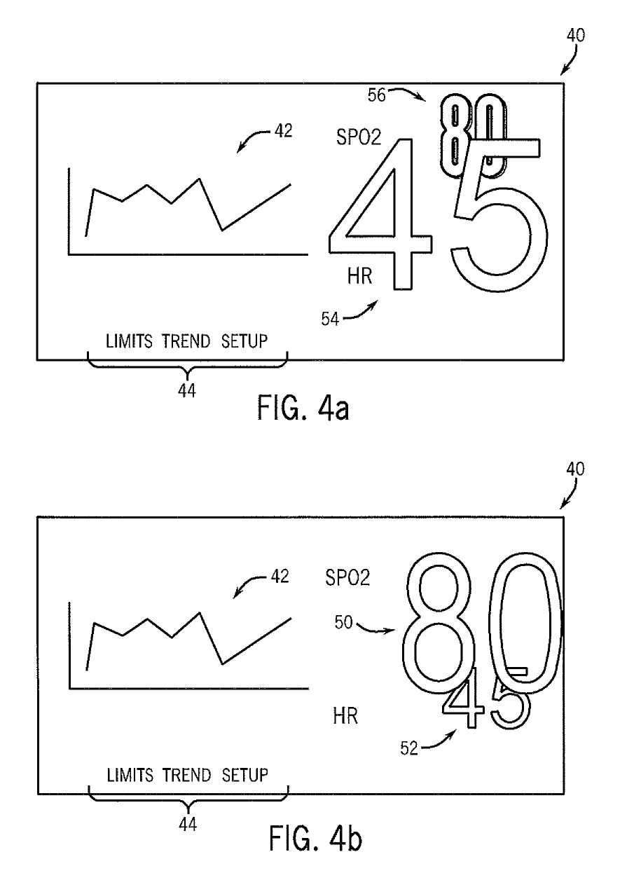

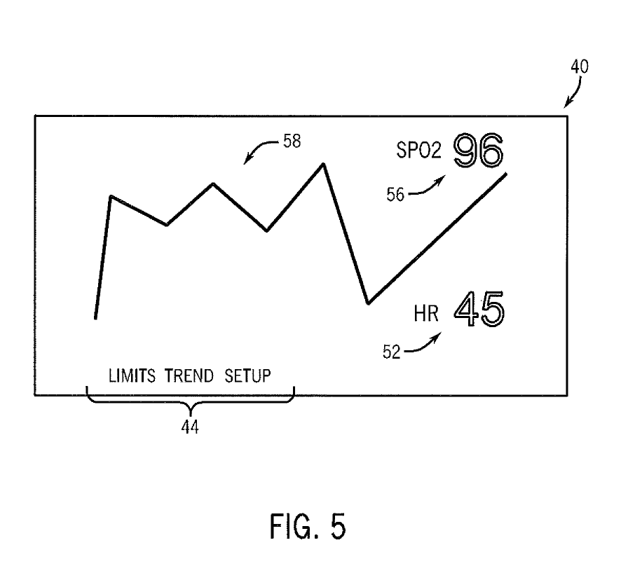

In certain embodiments, the SpO₂ measurement 50 is displayed with the enhanced visibility properties (e.g., larger font size) until the currently measured SpO₂ value returns to a normal level. That is, the display 40 will continue to display the SpO₂ measurement 50 at the larger font size, different font type, different font style, and/or different font color until the SpO₂alarm is no longer active. It is to be understood while the enhanced visibility properties of the SpO₂measurement 50 may stay the same during alarm activation, the displayed value (e.g., 80) will change in accordance to changes in the measured SpO₂ value. In other embodiments, the font size, font type, and font color of the SpO₂ measurement 50 may dynamically change while the alarm is still active. For example, the font size of the SpO₂ measurement 50 may cycle back and forth between a small font size and a large font size at a periodic time interval (e.g., 500 milliseconds, 1, 2, 3, 4, 10 seconds). The periodic cycling (i.e., flashing) of the font size may aid in procuring the attention of the clinician as well as in increasing situational awareness. Situational awareness is increased because the clinician is made aware that the alert condition still exists as long as the periodic cycling of the font size is still ongoing. Indeed, the disclosed techniques include a number of such dynamic visualizations, including those described in more detail with respect to FIGS. 4a, 4b, and 5 below, that can improve readability and situational awareness.

FIGS. 4a and 4b depict embodiments of certain visualizations presented by the display screen 40 when multiple alarms, such as a heart rate alarm and the SpO₂ alarm, have been activated. Accordingly, the display screen 40 may alternately present a more prominent heart rate measurement 54 as depicted in FIG. 4a, followed by the more prominent SpO₂ measurement 50, as depicted in FIG. 4b. As described below in more detail, the display screen 40 may cycle back and forth between the visualizations of FIG. 4a and the visualizations of FIG. 4b in order to present the multiple alarm measurements.

In the depicted embodiments of FIG. 4a, the heart rate measurement 54 has a larger font size, and may have a different font type, a different font style, and/or a different color. Additionally or alternatively, the SpO₂ measurement 56 may be displayed less prominently, for example, when compared to the SpO₂measurement 48 of FIG. 2. For example, the SpO₂ measurement 56 may be of a smaller size than the SpO₂ measurement 48 of FIG. 2. The less prominent display of the SpO₂ measurement 56 may include reducing the font size, increasing the font opacity (i.e., making the displayed value more transparent), changing the color, changing the font type, and/or changing the font style. In certain embodiments, the displayed heart rate measurement may periodically cycle back and forth between the small font heart measurement 46 and the large font heart measurement 54. At the expiration of a time interval, for example, four seconds, the visualizations of FIG. 4a may then transition to the visualizations of FIG. 4b. In other embodiments, the heart rate measurement may remain displayed at the large font measurement 54 throughout the time interval, for example, four seconds. At the expiration of the time interval the visualizations of FIG. 4a may subsequently transition to the visualizations described in more detail with respect to FIG. 4b below.

FIG. 4b depicts an embodiment of visualizations presented by the display screen 40 after the presentation of the visualizations of FIG. 4a. Accordingly, the second alarm measurement, such as the SpO₂ measurement 50, is displayed more prominently. Additionally or alternatively, the heart rate measurement 52 may be displayed less prominently than the heart rate measurement 46 of FIG. 2. For example, the heart rate measurement 52 may be of a smaller font size than the heart rate measurement 46 of FIG. 2. As mentioned previously, the SpO₂ measurement 50 has a larger font size, and may have a different font type, a different font style, and/or a different color. In certain embodiments, the displayed SpO₂ measurement may periodically cycle back and forth between the small font SpO₂ measurement 48 and the large font SpO₂measurement 50. At the expiration of a time interval, for example, four seconds, the visualizations of FIG. 4b may then transition to the visualizations of FIG. 4a. In other embodiments, the SpO₂ measurement may remain displayed at the large font measurement 50 throughout the time interval, for example, four seconds. At the expiration of the time interval the visualizations of FIG. 4b may subsequently transition to the visualizations described in more detail with respect to FIG. 4a above.

Indeed, the visualizations depicted in FIGS. 4a and 4b may periodically cycle back and forth between each other as long as there are multiple alarms currently active. Such a periodic cycling of visualizations is advantageous because it allows the clinician to easily read each of the multiple alarm measurements and to become situationally aware of the various alarms currently active. It is to be understood that any number of alarm measurement (e.g., patient alarms, system alarms) may be capable of participating in the visual display cycle. For example, if there are three or more active alarms, then the visual display cycle would include the display of the visuals for alarm one, followed by the visuals for alarm two, followed by the visuals for alarm three, and so forth. Indeed, any alarm measurement, such as the waveform depicted in FIG. 5, is able to participate in the visual display cycle.

FIG. 5 depicts an embodiment of certain visualizations presented by display screen 40 when the display of one or more alarm measurements includes a waveform or other measurement or trend graphic. Certain measurements, for example plethysmographic measurements, heart rate measurements, electrocardiogram measurements, electroencephalogram measurements, among others, may be visualized as a waveform. Accordingly, it may be useful to present a highly visible waveform, such as waveform 58. In certain embodiments, the waveform 58 includes a waveform of a larger size, for example a size 30%, 50%, 100%, 200%, larger than the size of the waveform 42 depicted in FIGS. 2, 3, 4a, 4b. In certain embodiments, the larger waveform 58 may remain at the same aspect ratio as the initial, smaller waveform 42. Additionally, the waveform 58 may be of a different color, increased line thickness, and/or decreased opacity than the smaller waveform 42.

As mentioned above, in one embodiment, other measurements that are currently being presented by the display screen 40, such as the heart rate measurement 52 and the SpO₂ measurement 56, may be presented less prominently. Accordingly, the measurements 52 and 56 may include a reduced font size, an increased font opacity, a different font type, a different font style, and/or a different color. For example, the font height may decrease from an initial height of approximately 10%, 20%, 30%, 40% of the screen 40 height to a vertical height of approximately 5%, 10%, 15%, 20% of the screen 40 height. In certain embodiments, the font width may decrease proportionally in order to maintain the same font aspect ratio. In other embodiments, the font aspect ration may change so as to improve readability and noticeability. Additionally, the font type, font style, opacity, and color may also change.

In certain embodiments, the waveform 58 is displayed with the enhanced visibility properties (e.g., larger waveform size) until the alarm value returns to a normal level. That is, the display 40 will continue to display the waveform 58 at the larger size, color, and/or line thickness while the waveform alarm is still active. It is to be understood while the waveform 58 may be displayed more prominently throughout the existence of the active alarm, the actual waveform shape will change in accordance to changes in the measured waveform values. In other embodiments, the larger size, color, and/or line thickness of the waveform 58 may periodically change while the alarm is still active. For example, the size, color, and/or line thickness of the waveform 58 may cycle back and forth between the smaller waveform 42 depicted in FIGS. 2, 3, 4a, 4b, and the larger waveform 58. The dynamic waveform visualization aids in procuring the attention of the clinician as well as in increasing situational awareness. Indeed, the visualizations of FIG. 5 may allow a clinician to easily view the depicted waveform from almost all locations of a patient's room. Other enhanced or enlarged displays of measurements may include, for example, a video display (e.g., ultrasound display), an image display, or a 3-dimensional (3D) display. As mentioned above, such displays may be enlarged to cover a larger area of the screen 40 and/or enhanced by changing colors, changing aspect ratios, displaying textual annotations, and so forth.

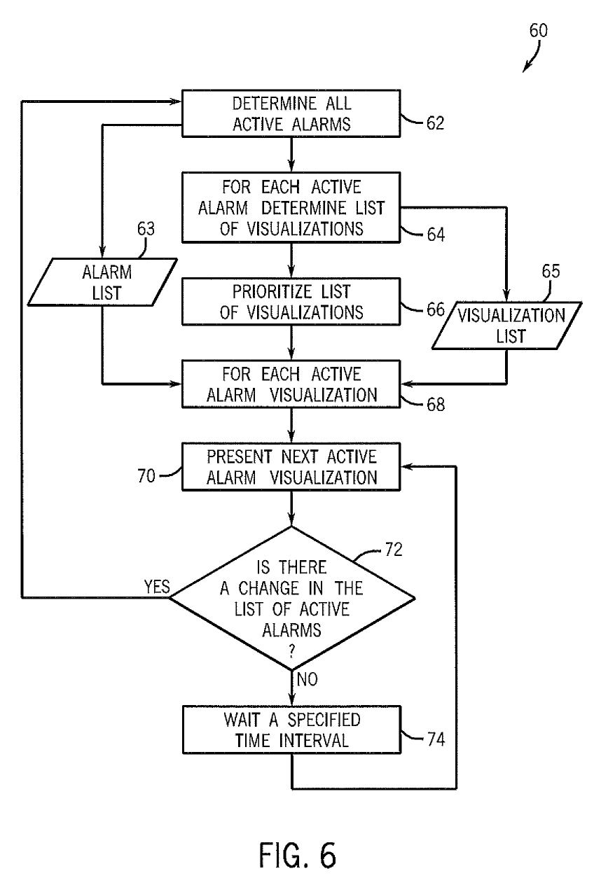

Turning to FIG. 6, the figure depicts a logic 60 that may be used, for example, by the processor of monitor 14 and/or the processor of multi-parameter monitor 24 to present one or more alarm visualizations. The processor following logic 60 determines all active alarms (block 62), for example, by comparing all configured alarm thresholds with the physiological measurements received via the sensor port 18 or with values derived from such sensor measurements. If a current measurement (e.g., patient measurement, system measurement) has crossed the configured alarm threshold, then the corresponding alarm is added to the active alarm list 63. If the measurement does not cross the configured threshold, then the corresponding alarm is not added to the active alarm list 63. Accordingly, a list of all active alarms is determined. The determination of all active alarms can be made very quickly, in some cases, within a few cycles of a microprocessor.

An individual alarm may have more than one alarm visualization. For example, the SpO₂ alarm may be configured by the clinician to include a plethysmographic waveform visualization (e.g., waveform 58) as well as a numeric text visualization (e.g., SpO₂ measurement 50). Accordingly, a list of all active visualizations 65 is determined (block 64) by determining the list of configured visualizations corresponding to each active alarm. In certain embodiments, the clinician is able to configure a list of alarm visualizations. That is, the clinician may configure an order of appearance for all alarm visualizations included in the patient monitoring system 10. For example, the first specified visualization may be the waveform 58, followed by the SpO₂ measurement 50, followed by the heart rate measurement 54, and so forth. Accordingly, the list of all current visualizations 65 may be prioritized and re-ordered (block 66) to correspond to the clinician's specified ordering.

The list of all active alarms 63 and all active visualizations 65 may then be used such that for each active alarm visualization (block 68), the visualization is first displayed (block 70), for example, for a given time interval (e.g., 1, 2, 3, 4, 5 seconds). That is, the logic 60 can iterate through each active alarm, and within each active alarm, present the active visualizations associated with the alarm. For example, suppose that there are two active alarms, alarm A and alarm B. Alarm A may include three active visualizations 1, 2, and 3, while alarm B may include two active visualizations, 3 and 4. Accordingly, visualization 1 may first be presented, followed by visualization 2, followed by visualization 3, followed by visualization 4, followed by visualization 5. As mentioned above with respect to FIGS. 3-5 the alarm visualizations may include larger waveforms, increased font sizes, different font types, different font styles, different opacity values, and so forth. Indeed, any of the aforementioned alarm visualizations may be presented, including visualizations that cycle from a smaller size to a larger size. After the first alarm visualization is presented (e.g., visualization 1), a decision is made to see if there is a change in the list of active alarms (decision 72). A change in the list of active alarms may occur, for example, due to a current measurement crossing outside of an alarm threshold (i.e., activating an alarm) or a current measurement falling within an alarm threshold (i.e., deactivating an alarm). If the list of active alarms remains unchanged, then the logic 60 may wait for a specified time interval (block 74). At the completion of the time interval, the presented alarm visualization transitions to the next alarm visualization (e.g., visualization 2) in the list of active alarm visualizations (block 70). If there is a change in the list of active alarms (decision 72), for example because a physiological measurement has activated a new alarm, then the logic 60 determines all active alarms (block 62) and the logic 60 continues with the next block 64 as described above.

The embodiments described above have been shown by way of example, and it should be understood that these embodiments may be susceptible to various modifications and alternative forms. It should be further understood that the claims are not intended to be limited to the particular forms disclosed, but rather to cover all modifications, equivalents, and alternatives falling within the spirit and scope of this disclosure.

What is claimed is:

1. A physiological monitor, comprising:
a display;
a sensor port configured to receive a physiological signal;
a processor in communication with the display and the sensor port, wherein the processor processes the physiological signal to generate a physiological measurement, compares the physiological measurement to an alarm threshold, and presents a visualization of the physiological measurement on the display at a first size when the physiological measurement is within the alarm threshold and present the visualization of the physiological measurement on the display at a second size when the physiological measurement is at or outside the alarm threshold.

2. The physiological monitor of claim 1, wherein the visualization of the physiological measurement comprises a textual display, waveform display, a video display, an image display, or a 3-dimensional (3D) display.

3. The physiological monitor of claim 1, wherein the processor displays the visualization on the display in a first color, font type, font style, or opacity when the alarm threshold is not exceeded and at a second color font type, font style, or opacity when the alarm threshold is exceeded.

4. The physiological monitor of claim 1, wherein the processor displays the visualization on the display at the second size at a periodic interval when the alarm threshold is exceeded.

5. The physiological monitor of claim 1, wherein the processor displays the visualization on the display at the second size for at least every half second (i.e., 500 milliseconds) when the alarm threshold is exceeded.

6. The physiological monitor of claim 4, wherein the periodic interval comprises at least half second (i.e., 500 milliseconds).

7. The physiological monitor of claim 1, wherein the second size is greater than the first size.

8. The physiological monitor of claim 1, wherein one or both of the first size and the second size are user configurable.

9. The physiological monitor of claim 1, wherein the processor executes stored code causing the processor to process the physiological signal, to compare the physiological measurement to the alarm threshold, and to display the visualization on the display.

10. A physiological monitor, comprising:
a display;
a sensor port; and
a processing component in communication with the display and the sensor port, wherein the processing component generates a physiological measurement based on input received via the sensor port, determines whether the physiological measurement meets an alarm condition, and alters the appearance of a visualization of the physiological measurement on the display when the physiological measurement meets the alarm condition such that the visualization can be read at a greater distance than when the appearance of the visualization is unaltered.

11. The physiological monitor of claim 10, wherein the visualization of the physiological measurement comprises a textual display, waveform display, a video display, an image display, or a 3-dimensional display.

12. The physiological monitor of claim 10, wherein the processing component determines whether the physiological measurement meets the alarm condition by comparing the physiological measurement to an alarm threshold.

13. The physiological monitor of claim 10, wherein the processing component alters the appearance of the visualization on the display by increasing a size used to display the visualization.

14. The physiological monitor of claim 10, wherein the processing component changes the color used to display the visualization on the display when the physiological measurement meets the alarm condition.

15. The physiological monitor of claim 10, wherein the processing component flashes the visualization having the altered appearance on the display at a periodic interval when the physiological measurement meets the alarm condition.

16. The physiological monitor of claim 10, wherein the processing component executes stored code causing the processing component to generate the physiological measurement, to determine whether the physiological measurement meets the alarm condition, and to alter the appearance of the visualization on the display when the physiological measurement meets the alarm condition.

17. A method for displaying one or more visualizations of physiological measurements, comprising:
displaying a first visualization of a first physiological measurement on a display at a first size when the first physiological measurement is within a first expected range; and
displaying the first visualization of the first physiological measurement on the display at a second size when the first physiological measurement is outside the expected range.

18. The method of claim 17, comprising,
displaying a second visualization of a second physiological measurement on the display at a third size when the second physiological measurement is within a second expected range; and
displaying the second visualization of the second physiological measurement on the display at a fourth size when the second physiological measurement is outside the expected range.

19. The method of claim 17, wherein the first visualization of the physiological measurement comprises a textual display, waveform display, a video display, an image display, or a 3-dimensional (3D) display.

20. The method of claim 18, wherein the first size is smaller than the second size, and the third size is smaller than the fourth size.
